(19) United States
(12) Patent Application Publication
(10) Pub. No.: US 2014/0024800 A1
(43) Pub. Date: Jan. 23, 2014

(54) CARBON DIOXIDE CAPTURE AND CONVERSION TO A CARBAMATE SALT AND POLYUREA

(71) Applicant: Qianli Chu, Grand Forks, ND (US)

(72) Inventor: Qianli Chu, Grand Forks, ND (US)

(73) Assignee: UNIVERSITY OF NORTH DAKOTA, Grand Forks, ND (US)

(21) Appl. No.: 13/550,943

(22) Filed: Jul. 17, 2012

(51) Int. Cl. C07C 227/02; C08G 71/02

(52) U.S. Cl. CPC C07C 227/02; C08G 71/02
USPC 528/310; 562/550

(57) ABSTRACT

A method for treating carbon dioxide includes preparing a polyamine composition and delivering a stream containing carbon dioxide to the polyamine composition. The carbon dioxide reacts with polyamine in the polyamine composition to form a carbamate salt. A method for producing polyurea from carbon dioxide includes delivering a stream containing carbon dioxide to a polyamine composition. The carbon dioxide reacts with polyamine in the polyamine composition to form a carbamate salt. The method also includes dehydrating the carbamate salt to produce polyurea.

### STATEMENT OF GOVERNMENT INTEREST

This invention was made with government support under Grant No. EPS 0814442 awarded by the National Science Foundation. The government has certain rights in the invention.

### BACKGROUND

Billions of tons of carbon dioxide are released into the Earth's atmosphere every year. Scientific studies have shown that greenhouse gases, including carbon dioxide, have contributed to global warming. Recently, efforts have been made to turn captured carbon dioxide into harmless and useful products. For example, commercially available carbon dioxide has been converted into carbonate polymers. However, this process requires a pure stream of carbon dioxide and expensive catalysts.

### SUMMARY

A method for treating carbon dioxide includes preparing a polyamine composition and delivering a stream of carbon dioxide to the polyamine composition. The carbon dioxide reacts with polyamine in the polyamine composition to form a carbamate salt.

A method for producing polyurea from carbon dioxide includes delivering a stream of carbon dioxide to a polyamine composition. The carbon dioxide reacts with polyamine in the polyamine composition to form a carbamate salt. The method also includes dehydrating the carbamate salt to produce polyurea.

### BRIEF DESCRIPTION OF THE DRAWINGS

FIG. 1 is a simplified flow diagram of a method for treating carbon dioxide.

FIG. 2 is a simplified flow diagram of a method for producing polyurea.

### DETAILED DESCRIPTION

According to the present invention, carbon dioxide from a gas stream is captured and the carbon dioxide is converted to a carbamate salt using a polyamine. The converted carbamate salt can be dehydrated to produce a polyurea, which can be used in the production of other polymer-containing materials such as spandex (elastane). No catalysts are used in the conversion of carbon dioxide to polyurea and no purification or processing of the stream containing carbon dioxide (e.g., flue stream) is required. The following chemical equation illustrates one embodiment of carbon dioxide capture and conversion to polyurea:

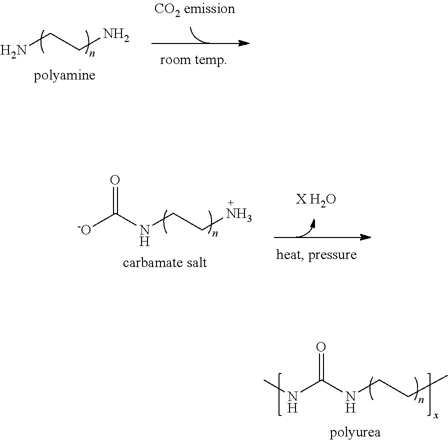

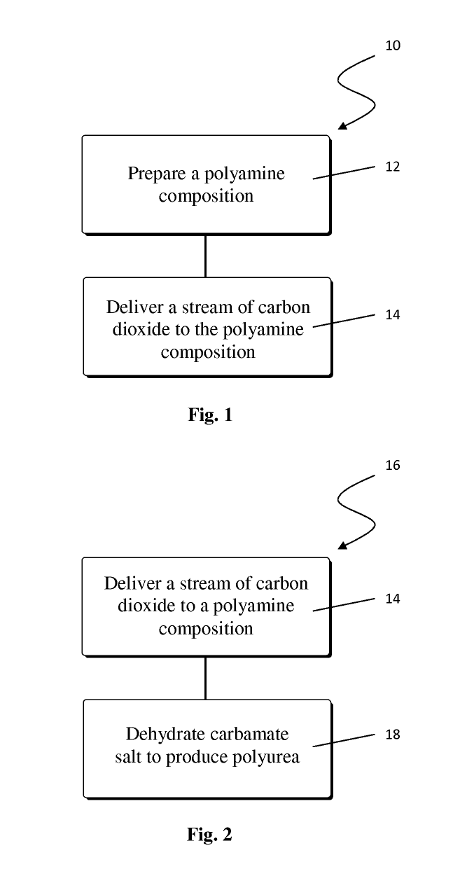

FIG. 1 shows a simplified flow diagram of a method for treating carbon dioxide. Method 10 includes preparing a polyamine composition (step 12) and delivering a stream of carbon dioxide to the polyamine composition (step 14). The carbon dioxide reacts with the polyamine composition to form a carbamate salt.

Polyamines contain two or more amino groups. The following is an example formula of a diamine, one type of polyamine:

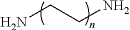

A polyamine composition is prepared in step 12 using one or more polyamines. Polyamines can be liquids or solids at room temperature, depending on the length of the carbon chain between the amino groups and any other functional groups present on the molecule. Polyamines that are solid at room temperature can be used to form a solid polyamine composition. Solid polyamine compositions are prepared so that a stream of gas containing carbon dioxide can be passed through the polyamine composition. Solid polyamine compositions include 1) polyamine particles or powder contained within screens or filters, 2) polyamine particles coated on walls of passages, ducts or filters and 3) any other configuration that allows polyamine particles to contact the gas stream in which the reacted polyamines can be later collected. In exemplary embodiments, the solid polyamine composition is prepared in such a way that the composition can be easily collected following step 14.

Polyamines that are liquids or solid at room temperature can be used to form a liquid polyamine composition. Like solid polyamine compositions, liquid polyamine compositions are prepared so that a stream of gas containing carbon dioxide can be passed through the polyamine composition. Liquid polyamine compositions can be aqueous or non-aqueous (e.g., liquid polyamine only, solution in ethanol, etc.). Liquid polyamine compositions are typically contained within a tank or other vessel through which the gas stream is delivered. In exemplary embodiments, vessels containing liquid polyamine compositions include mechanical mixing components for stirring the liquid polyamine composition. Suitable mechanical mixing components include mixing blades and other mechanical devices that increase the turbulence of the liquid polyamine composition.

Exemplary polyamines for use in the polyamine composition include hexamethylenediamine (H₂N(CH₂)₆NH₂), diethylenetriamine (H₂N(CH₂)₂NH(CH₂)₂NH₂), ethylenediamine (H₂N(CH₂)₂NH₂) and combinations thereof. Additional suitable polyamines include primary polyamines, secondary polyamines, tertiary polyamines, linear polyamines, branched polyamines, cyclic polyamines, and polyamines having aromatic or aliphatic rings and combinations of any of the above. The polyamine(s) with which the polyamine composition is prepared can be selected to produce particular carbamate salts, as described in greater detail below.

A gas stream containing carbon dioxide is delivered to the polyamine composition in step 14, where the carbon dioxide reacts with the polyamine composition to form a carbamate salt. In one example, the carbon dioxide and a diamine of the polyamine composition react according to the following reaction:

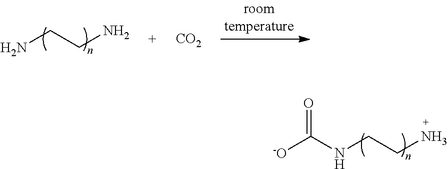

The gas stream delivered to the polyamine composition can contain various amounts of carbon dioxide. The gas stream does not need to be pure carbon dioxide. The gas stream can also contain other constituents such as nitrogen, oxygen, water vapor, nitrogen oxides, sulfur dioxide and trace elements. In exemplary embodiments, the gas stream will contain between about 0.03% and about 15% carbon dioxide. For example, suitable flue streams from many different kinds of power plants contain carbon dioxide at levels between approximately 5% and 12% by volume. However, the gas stream can contain up to 100% carbon dioxide or use air directly, which contains only about 0.039% carbon dioxide. The more carbon dioxide contained within the gas stream, the more quickly the reaction will occur. The gas stream can be a waste gas stream, such as a flue gas stream from the exhaust of a furnace, boiler, steam generator, power plant, kiln, cupola, or fermentation vessel.

The gas stream containing carbon dioxide is generally delivered to the polyamine composition between a temperature of about −20° C. (−4° F.) and about 400° C. (752° F.). In exemplary embodiments, the gas stream containing carbon dioxide is delivered to the polyamine composition at an ambient temperature between about 10° C. (50° F.) and about 55° C. (131° F.). Water, ice, air or other heat exchange systems can be used to cool the gas stream prior to delivery to the polyamine composition. If the gas stream has a temperature higher than the boiling point of the polyamines present in the polyamine composition, the polyamines can be vaporized and react with the carbon dioxide in the gas phase.

The gas stream can be delivered to the polyamine composition at virtually any pressure as long as the polyamine composition remains in place. In some embodiments, the gas stream is delivered to the polyamine composition at a pressure between about 100 kPa (14.5 psi) and about 1×10⁶ kPa (1.45×10⁵ psi).

The polyamine(s) of the polyamine composition reacts with the carbon dioxide according to the reaction shown above. The polyamine and carbon dioxide react to form a carbamate salt as a result of a nucleophilic addition and acid-base reaction between the polyamine and carbon dioxide. The amount of carbon dioxide that reacts with the polyamine can vary according to experiments performed. When a carbon dioxide emission was delivered to a pure solid, grinded hexamethylenediamine composition, nearly 1 mole of carbon dioxide was converted to carbamate salt for every 1 mole of hexamethylenediamine. When the polyamine composition was changed to an aqueous solution of hexamethylenediamine, nearly 1 mole of carbon dioxide was converted to carbamate salt for every 1 mole of hexamethylenediamine. According to additional experiments, the conversion rate and the rate of reaction were not affected by temperature. Depending on the scale of the polyamine composition, the gas stream flow rate and the amount of carbon dioxide present in the gas stream, complete conversion of the polyamine composition can occur in as few as 10 minutes.

The carbamate salt produced according to the reaction above is generally a white solid. The carbamate salt is separated from any unreacted polyamine composition and collected. Carbamate salts can be separated from unreacted polyamine composition by vaporization, filtration, precipitation, sieving and other methods of separation depending on the chemical and physical properties of the polyamine (i.e. solid or liquid, aqueous or not). The carbamate salt is easily handled, transported and stored, and sent off-site for further processing or processed further on-site. No catalysts are needed for the reaction between carbon dioxide and polyamines to produce the carbamate salt. In exemplary embodiments, the carbamate salt is an intermediate that is reacted to produce a polyurea, as described in greater detail below.

FIG. 2 shows a simplified flow diagram of a method for producing polyurea from carbon dioxide. Method 16 includes delivering a stream of carbon dioxide to a polyamine composition (step 14) where the carbon dioxide reacts with polyamine(s) in the polyamine composition to form a carbamate salt. Method 16 also includes dehydrating the carbamate salt to produce a polyurea in step 18.

A carbamate salt is dehydrated to produce polyurea according to the following reaction to produce polyurea:

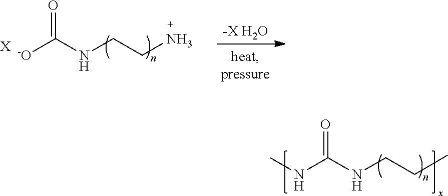

Polyurea is an elastomer that can be used to form polymeric coatings and synthetic fibers or films. For example, polyurea and polyurethane are used in the manufacture of spandex (elastane).

Carbamate salt dehydration occurs at a temperature between about 100° C. (212° F.) and about 450° C. (842° F.) at a pressure between about 1×10² kPa (14.5 psi) and about 1×10⁶ kPa (1.45×10⁵ psi). The dehydration reaction completes after a time between about 30 minutes/hours and about 48 hours. No catalysts are needed or used for the dehydration of the carbamate salt. In one exemplary embodiment, the carbamate salt is dehydrated at a temperature of about 370° C. (698° F.) at a pressure exceeding about 2758 kPa (400 psi) for about 6 hours. Under these conditions, polyurea was produced (from a carbamate salt formed from hexamethylenediamine) with a yield greater than 80%. The produced polyurea was a yellow polymer having high flexibility, high strength and a high melting temperature.

Depending on the polyamine chosen for the polyamine composition, the polyurea product can vary by polymer length (X) and carbon chain length (n). Additionally, functional groups on the carbon chain of the polyamine carry over to the carbamate salt and the polyurea. Thus, various functional groups can be added to the polyurea by incorporating those functional groups into the polyamine chosen for the polyamine composition.

Other acidic gases, such as sulfur dioxide, in the gas stream can also react with polyamines in a similar way as CO₂. Thus, those harmful gases can also be removed from the gas stream by this process. In exemplary embodiments, the gas stream contains about 0.05% of sulfur dioxide.

While the invention has been described with reference to exemplary embodiments, it will be understood by those skilled in the art that various changes may be made and equivalents may be substituted for elements thereof without departing from the scope of the invention. In addition, many modifications may be made to adapt a particular situation or material to the teachings of the invention without departing from the essential scope thereof. Therefore, it is intended that the invention not be limited to the particular embodiments disclosed, but that the invention will include all embodiments falling within the scope of the appended claims.

1. A method for treating carbon dioxide, the method comprising:
preparing a polyamine composition;
delivering a stream containing carbon dioxide to the polyamine composition, wherein the carbon dioxide reacts with polyamine in the polyamine composition to form a carbamate salt.

2. The method of claim 1, wherein the stream containing carbon dioxide is a waste stream from a power plant.

3. The method of claim 1, wherein the stream containing carbon dioxide is air.

4. The method of claim 1, wherein the polyamine composition comprises hexamethylenediamine.

5. The method of claim 1, wherein the polyamine composition comprises diethylenetriamine.

6. The method of claim 1, wherein the polyamine composition comprises a polyamine selected from the group consisting of primary polyamines, secondary polyamines, tertiary polyamines, linear polyamines, branched polyamines, cyclic polyamines, and polyamines having aromatic or aliphatic rings and combinations thereof.

7. The method of claim 1, wherein the polyamine composition comprises a solid polyamine.

8. The method of claim 1, wherein the polyamine composition comprises a liquid polyamine.

9. The method of claim 8, wherein the polyamine is in an aqueous solution.

10. The method of claim 8, wherein the polyamine is in an organic solution.

11. The method of claim 8, further comprising:
mechanically mixing the polyamine composition while delivering the stream of carbon dioxide to the polyamine composition.

12. The method of claim 1, further comprising:
separating the carbamate salt from the polyamine composition.

13. The method of claim 12, wherein the carbamate salt is separated from the polyamine composition by vaporization, filtration or precipitation.

14. The method of claim 1, wherein the stream of carbon dioxide is delivered to the polyamine composition at a temperature between about −20° C. and about 400° C.

15. The method of claim 1, wherein the stream of carbon dioxide is delivered to the polyamine composition at a pressure between about 100 kPa (14.5 psi) and about 1×10⁶ kPa (1.45×10⁵ psi).

16. The method of claim 1, further comprising:
dehydrating the carbamate salt to produce a polyurea.

17. The method of claim 16, wherein the method is performed in the absence of a catalyst.

18. The method of claim 16, wherein the carbamate salt is dehydrated at a pressure between about 1×10² kPa (14.5 psi) and about 1×10⁶ kPa (1.45×10⁵ psi).

19. The method of claim 16, wherein the carbamate salt is dehydrated at a temperature between about 100° C. and about 450° C.

20. The method of claim 16, wherein the carbamate salt is dehydrated for a time between about 30 minutes and about 48 hours.

21. A method for producing polyurea from carbon dioxide, the method comprising:
delivering a stream containing carbon dioxide to a polyamine composition, wherein the carbon dioxide reacts with polyamine in the polyamine composition to form a carbamate salt; and
dehydrating the carbamate salt to produce polyurea.

22. The method of claim 21, wherein the method is performed in the absence of a catalyst.
